(19) United States
(12) Patent Application Publication
(10) Pub. No.: US 2007/0156029 A1
(43) Pub. Date: Jul. 5, 2007

(54) Discernment of human health through electronic system input/output devices

(76) Inventors: Margaret E. Morris, Portland, OR (US); Terry J. Dishongh, Portland, OR (US); Anthony C. Salvador, Portland, OR (US)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(21) Appl. No.: 11/323,655

(22) Filed: Dec. 31, 2005

(51) Int. Cl. A61B 5/00

(52) U.S. Cl. 600/300; 128/920

(57) ABSTRACT

Methods and apparatuses for monitoring user interactions with an electronic device for indications of a degenerative condition.

### TECHNICAL FIELD

Embodiments of the invention relate to human health observation. More particularly, embodiments of the invention relate to techniques for discerning conditions in human health and taking action in response thereto.

### BACKGROUND

The “Age Wave”—a dramatic rise in lifespan and proportional increase in older adults—is a worldwide trend that mandates changes not only in the medical system but also in technology design and development. Computer systems and other electronic devices will certainly be affected by the needs of elderly users. Additionally, computing can help overcome current limitations in the detection of age related or other types of impairment. Cognitive impairment, for example, Alzheimer's disease, begs for new techniques to illuminate early disease markers.

Early detection of neurodegeneration is typically not possible because health care providers typically lack personal baseline data and subtle indicators of decline for an individual. Clinical criteria for diagnosis are simply too crude to catch early problems and the infrequent scheduling of clinical assessment prevents patterning of an individual's condition over short and long periods of time. Diagnosis may also be delayed by the tendency of individuals to avoid assessment that is not clearly associated with intervention (be it medication, behavioral or financial support). This delayed diagnosis has a huge opportunity cost: patients often miss an opportunity for aggressive treatment and we as a society lack data on early markers and disease trajectories.

### BRIEF DESCRIPTION OF THE DRAWINGS

Embodiments of the invention are illustrated by way of example, and not by way of limitation, in the figures of the accompanying drawings in which like reference numerals refer to similar elements.

FIG. 1 is a block diagram of one embodiment of an electronic system.

FIG. 2 is a flow diagram of one embodiment of a technique for monitoring user inputs that may indicate a degenerative condition.

FIG. 3a illustrates an original user interface configuration.

FIG. 3b illustrates a user interface configuration modified in response to monitored interaction characteristics.

### DETAILED DESCRIPTION

In the following description, numerous specific details are set forth. However, embodiments of the invention may be practiced without these specific details. In other instances, well-known circuits, structures and techniques have not been shown in detail in order not to obscure the understanding of this description.

Personal computing can be adapted to capture significant age related changes (e.g., in fine motor control, perception, orientation and coordination) while providing support to the end user. As one example, by analyzing cursor usage using Fitt's Law may provide data on early cognitive degeneration as well as facilitate continued computer throughout later life. Further with the early detection, apart from feedback there may be immediate benefit provided to the user. In one embodiment, supportive cues may be integrated into computing device functionality to enable continued usage and offered in a graduated, adaptive format. The degree of support required by a user may provide nuanced data to drive assessment and adaptive intervention.

Andres, R. O. and Hartug, K. J., “Predictions Of Head Movement Time Using Fitts' Law,” indicates that human interaction with a computer may be tracked through response times of various tasks. For example, a camera may be used to track eye movement and the acquired data may be used to generate a correlation based on Fitts' Law. The target may be moving or stationary for different types of assessment. As another example, a mouse may be fitted with a motor and/or gyroscope to provide active feedback that may also be used for assessment purposes.

Use of an algorithm such as Fitts' Law may be used for early detection of a degenerative disease. As described herein a cursor or other input mechanism may be used for diagnosis of a degenerative condition as well as for techniques to allow continued use of an associated electronic system (e.g., computer system, personal digital assistant, cellular telephone) as the degenerative condition progresses. Fitts' Law is a model of human psychomotor behavior developed in the 1950s based on time and distance. In general, Fitts' Law enables prediction of human movement and human motion based on rapid, aimed movement. Fitts discovered that movement time was a logarithmic function of distance when target size is held constant, and movement time was also a logarithmic function of target size when distance is held constant. Mathematically, Fitts' Law may be stated as:

$$MT = a + b {log}_{2} ( \frac{D}{W} + 1 )$$

where “MT” represents movement time, “a” and “b” are regression coefficients, “D” represents the distance of movement from start to target center and “W” represents the width of the target. Thus, there is a speed-accuracy tradeoff associated with pointing where targets that are smaller and/or farther from the starting point require more time to acquire.

Fitts' Law is a very successful and well-known model. Since the advent of graphical user interfaces, Fitts' Law has been applied to tasks where a cursor or other input indicator is positioned over a graphical target, for example, a button. Fitts' Law may be used to model both point-and-click actions and drag-and-drop actions.

The logarithm in Fitts' Law may be referred to as the Index of Difficulty (ID) for the target and may be measured, for example, in units of bits.

$$ID = {log}_{2} ( \frac{D}{W} + 1 )$$

$$MT = a + b ID$$

Units for “b” may be time/bit and “a” can be considered as incorporating reaction time and/or time required to click a button. The values used for “a” and “b” may change as the conditions under which the action occur change. For example, a mouse and a stylus may both be used for pointing, but may have different values for “a” and “b” associated with the actions.

An Index of Performance (IP) may be used to characterize how quickly pointing can be accomplished independent of the target involved. In general, IP may be defined in two ways: The first equation has the disadvantage of ignoring

$$IP = \frac{1}{a} or IP = \frac{{ID}_{average}}{{MT}_{average}} .$$

The first equation has the disavantage of ignoring the effects of “a” and the second equation has the disadvantage of using a potentially arbitrary value for ID_{average}. As described in greater detail below, Fitts' Law (or other modeling techniques) may be used to monitor user interactions with an electronic system to determine whether the user may meet criteria for a degenerative condition.

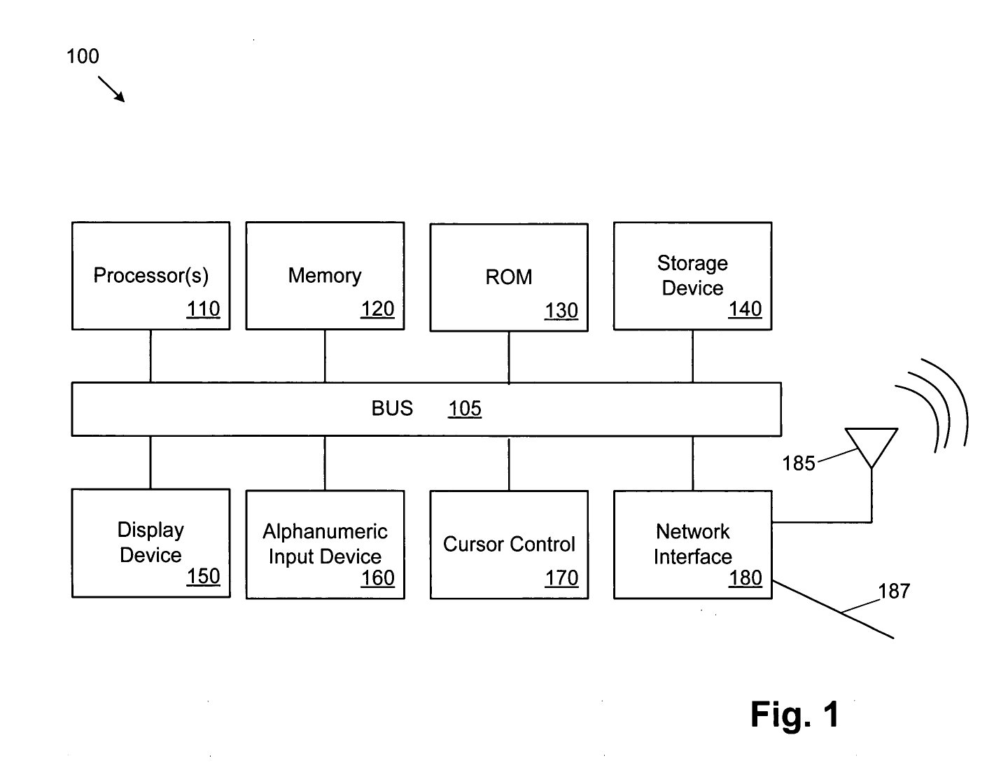

FIG. 1 is a block diagram of one embodiment of an electronic system. The electronic system illustrated in FIG. 1 is intended to represent a range of electronic systems (either wired or wireless) including, for example, desktop computer systems, laptop computer systems, cellular telephones, personal digital assistants (PDAs) including cellular-enabled PDAs, set top boxes. Alternative electronic systems may include more, fewer and/or different components.

Electronic system 100 includes bus 105 or other communication device to communicate information, and processor 110 coupled to bus 105 that may process information. While electronic system 100 is illustrated with a single processor, electronic system 100 may include multiple processors and/or co-processors. Electronic system 100 further may include random access memory (RAM) or other dynamic storage device 120 (referred to as memory), coupled to bus 105 and may store information and instructions that may be executed by processor 110. Memory 120 may also be used to store temporary variables or other intermediate information during execution of instructions by processor 110.

In one embodiment, memory 120 may include a software interaction analysis agent that may track user interactions (e.g., cursor movements, keystrokes) and apply Fitts' Law or other model to analyze the user interactions for signs of a degenerative condition. In alternate embodiments, the interaction analysis agent may be implemented in hardware, firmware or any combination of software, hardware and/or firmware.

Electronic system 100 may also include read only memory (ROM) and/or other static storage device 130 coupled to bus 105 that may store static information and instructions for processor 110. Data storage device 140 may be coupled to bus 105 to store information and instructions. Data storage device 140 such as a magnetic disk or optical disc and corresponding drive may be coupled to electronic system 100.

Electronic system 100 may also be coupled via bus 105 to display device 150, such as a cathode ray tube (CRT) or liquid crystal display (LCD), to display information to a user. In one embodiment, display device 150 may include an eye-tracking mechanism. Alphanumeric input device 160, including alphanumeric and other keys, may be coupled to bus 105 to communicate information and command selections to processor 110. Another type of user input device is cursor control 170, such as a mouse, a trackball, or cursor direction keys to communicate direction information and command selections to processor 110 and to control cursor movement on display 150.

The input/output devices, for example, display device 150, alphanumeric input device 160, cursor control 170, etc. may be used as described herein for diagnosis and/or compensation for degenerative conditions. In one embodiment, Fitts' Law, or other correlative technique, may be used monitor inputs from a user (e.g., cursor movement, keystroke data, eye tracking data) to determine whether a degenerative condition may exist in the user. In response, an indication of a diagnosis may be generated and/or outputs may be modified to compensate for the detected condition.

Electronic system 100 further may include network interface(s) 180 to provide access to a network, such as a local area network. Network interface(s) 180 may include, for example, a wireless network interface having antenna 185, which may represent one or more antenna(e). Network interface(s) 180 may also include, for example, a wired network interface to communicate with remote devices via network cable 187, which may be, for example, an Ethernet cable, a coaxial cable, a fiber optic cable, a serial cable, or a parallel cable.

In one embodiment, network interface(s) 180 may provide access to a local area network, for example, by conforming to IEEE 802.11b and/or IEEE 802.11g standards, and/or the wireless network interface may provide access to a personal area network, for example, by conforming to Bluetooth standards. Other wireless network interfaces and/or protocols can also be supported.

IEEE 802.11b corresponds to IEEE Std. 802.11b-1999 entitled “Local and Metropolitan Area Networks, Part 11: Wireless LAN Medium Access Control (MAC) and Physical Layer (PHY) Specifications: Higher-Speed Physical Layer Extension in the 2.4 GHz Band,” approved Sep. 16, 1999 as well as related documents. IEEE 802.11g corresponds to IEEE Std. 802.1-2003 entitled “Local and Metropolitan Area Networks, Part 11: Wireless LAN Medium Access Control (MAC) and Physical Layer (PHY) Specifications, Amendment 4: Further Higher Rate Extension in the 2.4 GHz Band,” approved Jun. 27, 2003 as well as related documents. Bluetooth protocols are described in “Specification of the Bluetooth System: Core, Version 1.1,” published Feb. 22, 2001 by the Bluetooth Special Interest Group, Inc. Associated as well as previous or subsequent versions of the Bluetooth standard may also be supported.

In addition to, or instead of, communication via wireless LAN standards, network interface(s) 180 may provide wireless communications using, for example, Time Division, Multiple Access (TDMA) protocols, Global System for Mobile Communications (GSM) protocols, Code Division, Multiple Access (CDMA) protocols, and/or any other type of wireless communications protocol.

In one embodiment, the following application of Fitts' Law may be used for monitoring of inputs. A movement time (MT) corresponding to the time required to complete a task successfully may be considered a ratio of an index of difficulty (ID) corresponding to the task and an index of performance (IP). In one embodiment the index of performance may be computed based on a number of misses and retries for the task. In one embodiment, the following equation may be used:

$$MT = \frac{ID}{IP}$$

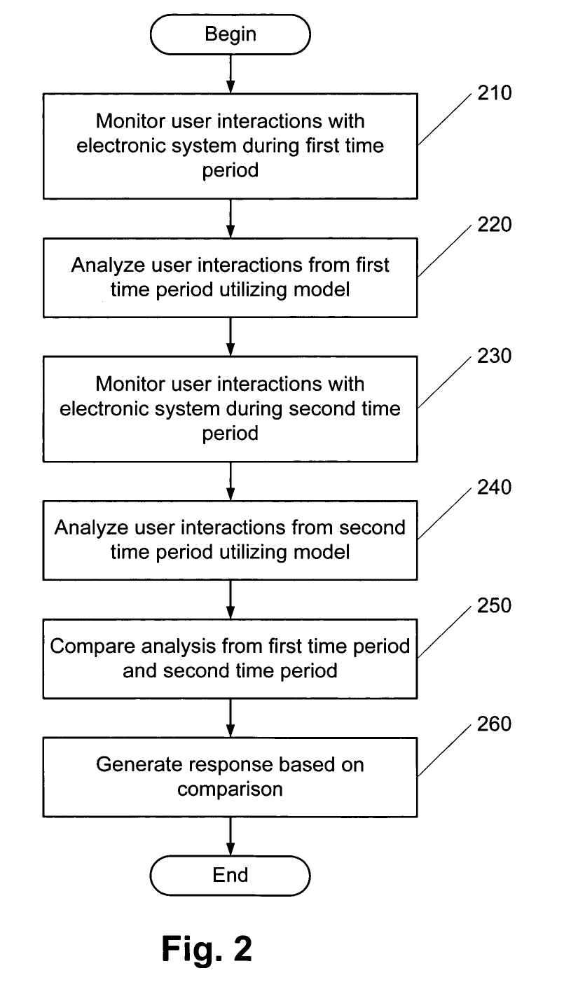

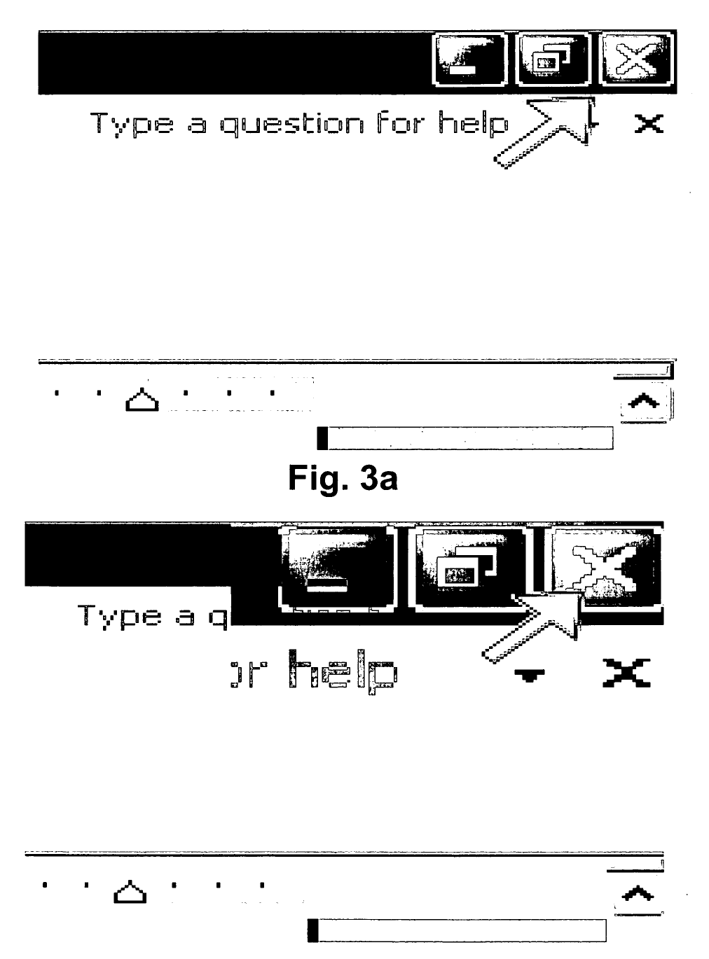

FIG. 2 is a flow diagram of one embodiment of a technique for monitoring user inputs that may indicate a degenerative condition. The example of FIGS. 2, 3a and 3b is described in terms of a person interacting with a computer system using a mouse to move a cursor displayed on a display device. However, the techniques described herein are applicable to many types of human interaction with an electronic system that has the ability to monitor the interaction including, for example, a personal digital assistant, a tablet computing device, cellular telephone, etc.

User interactions with a computer system (or other electronic device) during a first period of time may be monitored by the system, 210. The interactions may include, for example, movements of a cursor or pointer with a mouse, trackpad, trackball, joystick, etc. Monitoring of the interactions may include monitoring of any characteristics of the interaction including, but not limited to, distance of movement, speed of movement, accelerations, decelerations, selection location (e.g., cursor/pointer location at the time of a button activation), distance of the selection location from a target (e.g., radio button of a dialog box), eye movements, biofeedback, or any combination thereof.

In one embodiment, monitoring of user interaction may be associated with a user account to increase the likelihood that the monitored interactions correspond to a specific user. Other techniques may be used to determine a user identity, for example, a user name may be requested. As another example, characteristics associated with a user, for example, style of cursor movement or recognizable user characteristics.

The characteristics of the interaction may be monitored for a period of time in order to acquire a set of data for analysis that can be used for later comparison. The period of time may be any desired period of time, for example, 10 minutes, one day, one computing session, use of a selected program over a period of time, etc. Data corresponding to the monitored interaction may be stored in any suitable manner and/or format for analysis.

The characteristics of the interaction corresponding to the first period of time may be analyzed, 220. Analysis may be accomplished using any appropriate modeling technique. In one embodiment, Fitts' Law may be utilized as the modeling technique used with the characteristics of the interactions during the first period of time.

User interactions with the computer system during a second period of time may be monitored by the system, 230. The characteristics may be monitored in the same manner as described above with respect to the first period of time. The characteristics of the interaction corresponding to the second period of time may be analyzed, 240. Analysis may be accomplished using any appropriate modeling technique. In one embodiment, Fitts' Law may be utilized as the modeling technique used with the characteristics of the interactions during the second period of time.

The results of the analysis corresponding to the first period of time may be compared with the results of the analysis corresponding to the second period of time, 250. In one embodiment, using Fitts' Law, if the movement time (MT) varies by a threshold value, the difference between the first interaction and the second interaction may be considered significant. Using different modeling techniques different characteristics may be monitored and/or analyzed. Different thresholds may be used for different characteristics. Modeling may be accomplished using different modeling techniques for different characteristics corresponding to common periods of time. This may provide a more complex and possibly more accurate understanding of the user interactions.

In response to the comparison, a response may be generated, 260. The response may include, for example, magnification of a portion of a graphical user interface, modification of cursor/pointer movement, notification to the user that symptoms of a degenerative condition have been detected, notification to a third party (e.g., a doctor, a care provider) that symptoms of a degenerative condition have been detected, or any combination thereof.

The response that is generated may be based, at least in part, on the characteristics, or symptoms detected. That is, if a user exhibits decreased eye-hand coordination or decreased eyesight, a portion of the user interface may be, for example, enlarged. If a user exhibits symptoms of color-blindness, a color scheme used with the graphical user interface may be modified. Other responses may also be supported based, at least in part, on the characteristics or symptoms detected in the user.

FIG. 3a illustrates an original user interface configuration. The example of FIGS. 3a and 3b are directed to the “Minimize,” “Restore” and “Close” buttons found on many Microsoft Office products; however, the techniques described herein are equally applicable to other components of a graphical user interface. In order to exit an application a user may select the Close button by moving a pointer using a cursor control device (e.g., mouse, trackpad, trackball) to the location of the button and clicking a button.

However, if the user is suffering from a degenerative condition that may be detected as described herein, the user may have difficulty causing the point to move to the location of the desired button. Various actions may be taken in response to detecting a difficulty in moving the pointer to the button. In one embodiment, the size of buttons on the projected path of the pointer may be enlarged. In another embodiment, the pointer may be moved toward a button more directly tan the user may be able to accomplish using standard cursor control techniques alone. Other actions may also be taken.

FIG. 3b illustrates a user interface configuration modified in response to monitored interaction characteristics. The example of FIG. 3b illustrates but one example modification that may be utilized in response to detecting a degenerative condition in a user. If the pointer appears to be directed to a particular location on a graphical user interface, buttons or other input options may be enlarged. In the example of FIG. 3b, the Minimize, Restore and Close buttons are enlarged. Other portions of the graphical user interface may be enlarged as well.

Reference in the specification to “one embodiment” or “an embodiment” means that a particular feature, structure, or characteristic described in connection with the embodiment is included in at least one embodiment of the invention. The appearances of the phrase “in one embodiment” in various places in the specification are not necessarily all referring to the same embodiment.

While the invention has been described in terms of several embodiments, those skilled in the art will recognize that the invention is not limited to the embodiments described, but can be practiced with modification and alteration within the spirit and scope of the appended claims. The description is thus to be regarded as illustrative instead of limiting.

What is claimed is:

1. A method comprising:
monitoring user interactions with an electronic device;
analyzing the interactions based, at least in part, on characteristics of one or more human degenerative physical conditions; and
generating a human-observable response based, at least in part, on results of the analysis.

2. The method of claim 1 wherein generating the human-observable response based, at least in part, on the results of the analysis comprises modifying a user interface to compensate for the detected human degenerative physical condition.

3. The method of claim 2 wherein the modification of the user interface comprises enlarging a selected portion of the user interface.

4. The method of claim 2 wherein the modification of the user interface comprises modifying a cursor based, at least in part on the results of the analysis.

5. The method of claim 1 wherein generating the human-observable response based, at least in part, on the results of the analysis comprises sending a human-readable notification having an indication of the human degenerative physical condition.

6. The method of claim 1 wherein analyzing the interactions based, at least in part, on characteristics of one or more human degenerative physical conditions comprise applying Fitts' Law to the monitored user interactions.

7. The method of claim 1 wherein monitoring user interactions with the electronic device comprises:
monitoring input signals to the electronic device to control an indicator to be displayed on a display device coupled with the electronic device;
determining a corresponding indicator position on the display device; and
comparing the indicator position with potential targets displayed on the display device.

8. The method of claim 7 wherein the indicator comprises one of a cursor and/or a pointer.

9. An article comprising a computer-readable medium having stored thereon instructions that, when executed, cause one or more processors to:
monitor user interactions with an electronic device;
analyze the interactions based, at least in part, on characteristics of one or more human degenerative physical conditions; and
generate a human-observable response based, at least in part, on results of the analysis.

10. The article of claim 9 wherein the instructions that cause the one or more processors to generate the human-observable response based, at least in part, on the results of the analysis comprise instructions that, when executed, cause the one or more processors to modify a user interface to compensate for the detected human degenerative physical condition.

11. The article of claim 10 wherein the instructions that cause the modification of the user interface comprise instructions that, when executed, cause the one or more processors to enlarge a selected portion of the user interface.

12. The article of claim 10 wherein the instructions that cause the modification of the user interface comprise instructions that, when executed, cause the one or more processors to modify a cursor based, at least in part on the results of the analysis.

13. The article of claim 9 wherein the instructions that cause the one or more processors to generate the human-observable response based, at least in part, on the results of the analysis comprise instructions that, when executed, cause the one or more processors to send a human-readable notification having an indication of the human degenerative physical condition.

14. The article of claim 9 wherein the instructions that cause the one or more processors to analyze the interactions based, at least in part, on characteristics of one or more human degenerative physical conditions comprise instructions that, when executed, cause the one or more processors to apply Fitts' Law to the monitored user interactions.

15. The article of claim 9 wherein the instructions that cause the one or more processors to monitor user interactions with the electronic device comprise instructions that, when executed, cause the one or more processors to:
monitor input signals to the electronic device to control an indicator to be displayed on a display device coupled with the electronic device;
determine a corresponding indicator position on the display device; and
compare the indicator position with potential targets displayed on the display device.

16. The article of claim 15 wherein the indicator comprises one of a cursor and/or a pointer.

17. An apparatus comprising:
an input interface to receive input signals from one or more user input devices;
an output interface to provide output signals to at least one output device;
an interaction analysis agent communicatively coupled with the input interface and the output interface to monitor user interactions with an electronic device as indicated by at least the input signals, analyze the interactions based, at least in part, on characteristics of one or more human degenerative physical conditions, and cause to be generated a human-observable response based, at least in part, on results of the analysis.

18. The apparatus of claim 17 wherein generating the human-observable response based, at least in part, on the results of the analysis comprises generating signals to cause a graphical user interface to be modified to compensate for the detected human degenerative physical condition.

19. The apparatus of claim 18 wherein the modification of the graphical user interface comprises enlarging a selected portion of the user interface.

20. The apparatus of claim 18 wherein the modification of the user interface comprises modifying a cursor based, at least in part on the results of the analysis.

21. The apparatus of claim 17 wherein generating the human-observable response based, at least in part, on the results of the analysis comprises sending a human-readable notification having an indication of the human degenerative physical condition.

22. The apparatus of claim 17 wherein analyzing the interactions based, at least in part, on characteristics of one or more human degenerative physical conditions comprise applying Fitts' Law to the monitored user interactions.

23. A system comprising:
a cursor control mouse;
an input interface to receive input signals from the mouse;
an output interface to provide output signals to at least one output device;
an interaction analysis agent communicatively coupled with the input interface and the output interface to monitor user interactions with an electronic device as indicated by at least the input signals, analyze the interactions based, at least in part, on characteristics of one or more human degenerative physical conditions, and cause to be generated a human-observable response based, at least in part, on results of the analysis.

24. The system of claim 23 wherein generating the human-observable response based, at least in part, on the results of the analysis comprises generating signals to cause a graphical user interface to be modified to compensate for the detected human degenerative physical condition.

25. The system of claim 24 wherein the modification of the graphical user interface comprises enlarging a selected portion of the user interface.

26. The system of claim 24 wherein the modification of the user interface comprises modifying a cursor based, at least in part on the results of the analysis.

27. The system of claim 23 wherein generating the human-observable response based, at least in part, on the results of the analysis comprises sending a human-readable notification having an indication of the human degenerative physical condition.

28. The system of claim 23 wherein analyzing the interactions based, at least in part, on characteristics of one or more human degenerative physical conditions comprise applying Fitts' Law to the monitored user interactions.
